(19) United States
(12) Patent Application Publication
(10) Pub. No.: US 2023/0226370 A1
(43) Pub. Date: Jul. 20, 2023

(54) FEEDBACK ENERGY-RELEASE SYSTEM AND OPERATION METHOD THEREOF

(71) Applicant: iBeauty Biotechnology Company, Taipei City (TW)

(72) Inventor: Tsung-Hua Tsai, Taipei City (TW)

(73) Assignee: iBeauty Biotechnology Company, Taipei City (TW)

(21) Appl. No.: 18/190,069

(22) Filed: Mar. 25, 2023

Related U.S. Application Data
(63) Continuation of application No. 16/427,339, filed on May 30, 2019.

(30) Foreign Application Priority Data
Jun. 1, 2018 (TW) 107118836

(51) Int. Cl. A61N 5/06; A61N 7/00; A61N 2/02; G16H 40/63; G06N 20/00

(52) U.S. Cl. CPC A61N 5/0616; A61N 7/00; A61N 2/02; G16H 40/63; G06N 20/00; A61N 2/004

(57) ABSTRACT

A feedback energy-release system and operation method thereof are provided, adapted to provide an energy parameter to an energy-release apparatus for performing a plurality of energy-release operations on a target skin. A feature capturing device captures tissue feature data of the target skin, wherein the tissue feature data captured before and after each energy-release operation are a first and a second tissue feature data, respectively. A control device compares the second tissue feature data with a plurality of reference tissue feature data to obtain at least one suggested energy parameter, determines grades of the tissue feature data of the current energy-release operation and records a grade distribution of the tissue feature data. The control device generates the energy parameter of the next energy-release operation according to a difference between the first and second tissue feature data, the at least one suggested energy parameter and the grade distribution.

### CROSS-REFERENCE TO RELATED APPLICATION

This application is a continuation application of and claims the priority benefit of U.S. application Ser. No. 16/427,339, filed on May 30, 2019, now pending, which claims the priority benefit of Taiwan application serial no. 107118836, filed on Jun. 1, 2018. The entirety of each of the above-mentioned patent applications is hereby incorporated by reference herein and made a part of this specification.

### BACKGROUND

### Technical Field

The invention relates to an energy release control technique, and more particularly to a feedback energy-release system and an operation method thereof.

### Description of Related Art

A general way of skin receiving energy is handled by a user operating an energy-release apparatus to release specific energy on a target skin of the object, such as light, sound waves, electromagnetic waves, and the like. When the user operates the energy-release apparatus, it is often necessary to determine the energy parameter by experience. For example, the user visually checks the state of the target skin (e.g., telangiectasia, hemangioma, varicose veins, freckles, age spots, black lice and/or hair) to determine the intensity, the wavelength, time or range of the applied energy, otherwise the user operates directly according to the standard parameters provided by the manufacturer at the beginning and cannot adapt to the target skin of different objects and different conditions. After the skin receives the energy, the user then visually monitors the state changing of the target skin and needs to manually adjust the energy parameters based on the experience accumulated by himself/herself. In this way, it may not be effective enough and increase the number of energy-release operations or increase the possibility of side effects, thus it may require multiple trial and error learning to gradually achieve the desired results. In addition, when a user operates the energy-release apparatus improperly, there may be problems such as burning skin and other side effects or poor results.

### SUMMARY

In view of the above technical problems, embodiments of the present invention provide a feedback energy-release system and an operation method thereof, which can provide the user with objective energy parameters, and the provided energy parameter can be automatically adjusted to the state of the target skin. The functions of improving efficiency, processing effect and safety can be achieved.

An embodiment of the invention provides a feedback energy-release system adapted to provide an energy parameter to an energy-release apparatus, wherein the energy-release apparatus performs a plurality of energy-release operations on a target skin of an energy-release object. The feedback energy-release system comprises a feature capturing device and a control device.

The feature capturing device is configured to capture a plurality of tissue feature data of the target skin, wherein the tissue feature data, captured by the feature capturing device, before and after each of the energy-release operations are a first tissue feature data and a second tissue feature data, respectively, wherein the feature capturing device further captures the tissue feature data at different time points during the current energy-release operation. The control device is coupled to the feature capturing device and the energy-release apparatus. The control device receives the tissue feature data from the feature capturing device and compares the second tissue feature data with a plurality of reference tissue feature data to obtain at least one suggested energy parameter. The control device determines grades of the tissue feature data of the current energy-release operation, records a grade distribution of the tissue feature data of the current energy-release operation, and generates the energy parameter of the next energy-release operation according to a difference between the first tissue feature data and the second tissue feature data, the at least one suggested energy parameter and the grade distribution.

An embodiment of the invention provides an operation method of a feedback energy-release system, adapted to provide an energy parameter to an energy-release apparatus, wherein the energy-release apparatus performs a plurality of energy-release operations on a target skin of an energy-release object. The operation method comprising the following steps: capturing a plurality of tissue feature data of the target skin, wherein the tissue feature data captured before and after each of the energy-release operations are a first tissue feature data and a second tissue feature data, respectively, wherein the step of capturing the plurality of tissue feature data of the target skin comprises the step of capturing the tissue feature data at different time points during the current energy-release operation; comparing the second tissue feature data with a plurality of reference tissue feature data to obtain at least one suggested energy parameter; determining grades of the tissue feature data of the current energy-release operation and recording a grade distribution of the tissue feature data of the current energy-release operation; and generating the energy parameter of the next energy-release operation according to a difference between the first tissue feature data and the second tissue feature data, the at least one suggested energy parameter and the grade distribution.

Based on the above, the feedback energy-release system and the operation method thereof according to the embodiments of the present invention can automatically and efficiently provide objective suggested energy parameters. By comparing the difference of the tissue feature data captured before and after the energy-release operation, the feedback energy-release system estimates the effect of the last energy-release operation so as to automatically generate an energy parameter of the next energy-release operation to avoid the uncertainty of the user only by empirical operation. Moreover, through the reference database, more objective reference tissue feature data and corresponding reference energy parameters can be collected to provide more accurate and effective suggested energy parameters.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a block diagram illustrating a feedback energy-release system in accordance with an embodiment of the present invention.

FIG. 2 is a flow chart illustrating an operation method of the feedback energy-release system of FIG. 1 of the present invention.

FIG. 3 is a block diagram illustrating a control device according to the embodiment of FIG. 1 of the present invention.

FIG. 4 is a schematic diagram illustrating a network architecture of a feedback energy-release system in accordance with an embodiment of the present invention.

FIG. 5A is a schematic diagram illustrating a feedback energy-release system in accordance with another embodiment of the present invention.

FIG. 5B is a schematic diagram illustrating a feedback energy-release system in accordance with another embodiment of the present invention.

FIG. 6 is a flow chart illustrating an operation method of the feedback energy-release system in accordance with an embodiment of the present invention.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

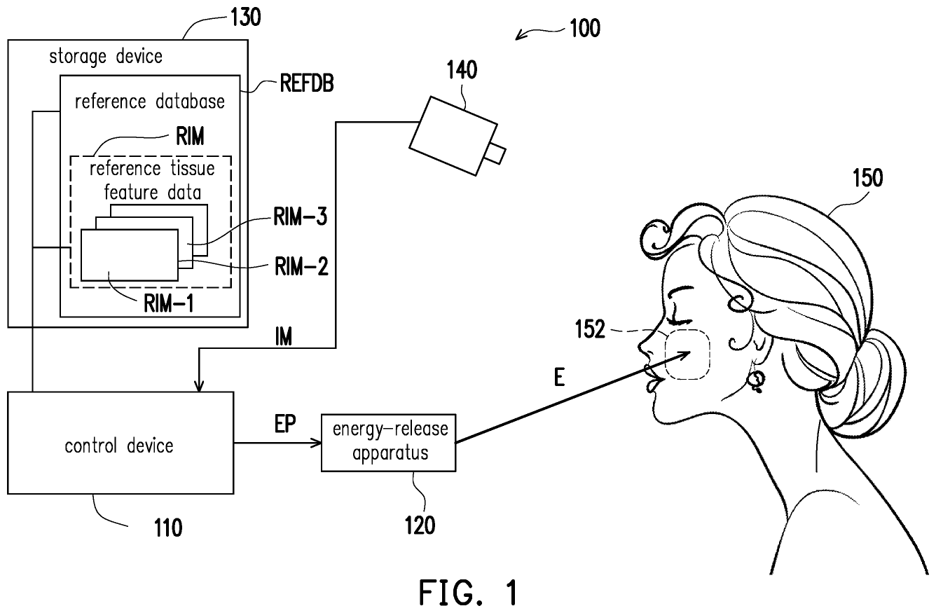

FIG. 1 is a block diagram illustrating a feedback energy-release system in accordance with an embodiment of the present invention. Referring to FIG. 1, an energy-release apparatus 120 releases a specific energy E to the target skin 152 of the energy-release object 150 for performing multiple energy-release operations. The feedback energy-release system 100 is adapted to provide an energy parameter EP to the energy-release apparatus 120 to control the energy E. The energy-release apparatus 120 performs the energy-release operation on the target skin 152 based on the energy parameter EP.

The feedback energy-release system 100 includes a control device 110 and a feature capturing device 140. The control device 110 is coupled to the feature capturing device 140 and the energy-release apparatus 120. The feature capturing device 140 is configured to capture a plurality of tissue feature data IM of the target skin 150. The tissue feature data IM captured by the feature capturing device 140 before each of the energy-release operations is referred to as a first tissue feature data, and the tissue feature data IM captured after each of the energy-release operations is referred to as a second tissue feature data. The control device 110 receives the tissue feature data IM from the feature capturing device 140 and provides the energy parameter EP to the energy-release apparatus 120.

In the embodiment, the feedback energy-release system 100 further includes a reference database REFDB, which is coupled to the control device 110 and has a plurality of reference energy parameters and a plurality of reference tissue feature data RIM, including RIM-1, RIM-2, RIM-3, etc. The plurality of reference energy parameters have a first correspondence with the plurality of reference tissue feature data RIM.

In the embodiment, the control device 110 can compare the current tissue feature data IM of the target skin 152 with the plurality of reference tissue feature data RIM to obtain at least one energy suggestion parameter. Specifically, after performing an energy-release operation, the control device 110 can compare the current tissue feature data IM of the target skin 152, also as the second tissue feature data, with the reference tissue feature data RIM. The control device 110 finds a reference tissue feature data, such as the reference tissue feature data RIM-2, matching with the second tissue feature data among the plurality of reference tissue feature data RIM, and provides the reference energy parameter corresponding to the matched reference tissue feature data RIM-2 as a suggested energy parameter according to the first correspondence.

The control device 110 may further compare the changes of the target skin 152 before and after the energy-release operation, that is, to compare the first tissue feature data with the second tissue feature data. The control device 110 generates the energy parameter EP of the next energy-release operation according to a difference between the first tissue feature data and the second tissue feature data and the suggested energy parameter described above.

It should be noted that the energy-release object 150 is exemplified by a female in FIG. 1, but the energy-release object 150 is not limited to age or gender.

Hereinafter, exemplary embodiments of the present invention will be described in detail.

Specifically, the control device 110 may be a personal computer, an ultra-mobile personal computer (UMPC), a work station, a server, or other electronic devices, and is not limited to the above.

The feature capturing device 140 may include an image capturing device such as a camera, other optical image capturing device, an infrared camera, an ultrasonic scanning device, a magnetic resonance imaging (MRI) machine, an optical coherence tomography (OCT) scanner and so on. The feature capturing device 140 may also include a skin physiological data acquisition equipment such as a skin detector or a temperature sensing device, or a combination of the above equipment, but is not limited thereto.

When the feature capturing device 140 includes an image capturing device, the tissue feature data IM includes, for example, an optical image, an infrared thermal image, an ultraviolet light image, an optical coherence tomography image or an ultrasonic image, or other types of images. These images, for example, include at least one of color, depth, shape, area, and thickness of spots, lines, pigments, blood vessels, hair, epidermis, dermis layer or subcutaneous tissue. When the feature capturing device 140 includes a skin physiological data acquisition equipment, for example, the feature capturing device 140 is a skin detector.

In an embodiment, besides capturing the tissue feature data IM of the target skin 152 before or/and after each energy-release operation, the feature capturing device 140 may also track the target skin 152 during the energy-release operation, or the user manually determines at which time point the feature capturing device 140 captures the tissue feature data IM of the target skin 152.

The energy E released by the energy-release apparatus 120 is in the form of laser, an electromagnetic wave, an ultrasonic wave or a pulsed light, and the form of the energy E is not intended to limit the present invention. The energy parameter EP includes the intensity, a wavelength, a waveform, a bandwidth, a length of energy release time, density, spot size of the energy E, the area where the target skin 152 receives energy, and the depth of the energy E penetrating the target skin 152, or other parameters used to control the energy E, and so on.

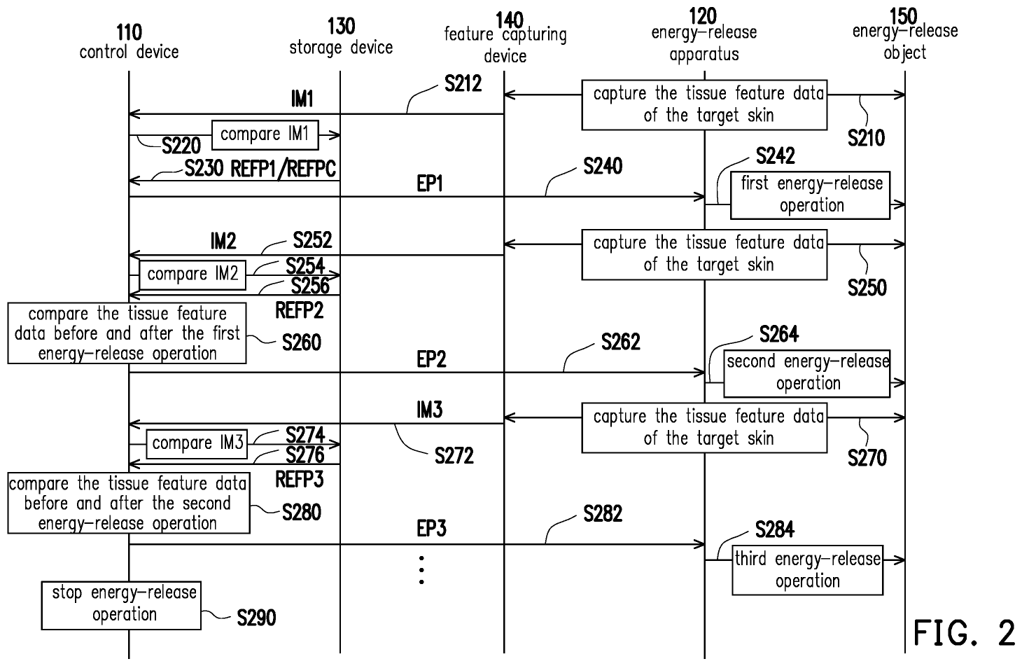

FIG. 2 is a flow chart illustrating an operation method of the feedback energy-release system of FIG. 1 of the present invention. Referring to FIG. 2 in conjunction with FIG. 1, the operation method of the feedback energy-release system 100 will be described in detail below.

In the present embodiment, the feedback energy-release system 100 further includes a storage device 130, storing the above-mentioned reference database REFDB. The control device 110 can be coupled to the storage device 130 through a wire/wireless network. The storage device 130 can be a cloud server, or a separate storage medium (such as an external hard disk, a flash drive, a cache memory or a memory card) that can be combined or separated from the control device 110, or is integrated with the control device 110 into an integrated storage device (for example, a hard disk), and the present invention does not limit the form of the storage device 130.

Here, the storage device 130 takes cloud storage as an example.

In the embodiment, in addition, to have the first correspondence with the reference tissue feature data RIM, the reference energy parameter stored in the reference database REFDB further has a second correspondence with the personal characteristics. The above personal characteristics include at least one of race, gender, age, skin color, skin type, place of residence, degree of sun exposure, medication record, type of skin problems, the extent of skin problems, treatment of skin problems, and reactions of the treatments. The above skin types are, for example, dry skin, oily skin, combination skin, normal skin, sensitive skin or congenital allergic skin, and the type of skin problem is, for example, the type of lesion.

In the embodiment of FIG. 2, before the target skin 152 receives any of the energy-release operations, steps S210 and S212 are performed, the feature capturing device 140 scans, photographs or detects the target skin 152 to generate the tissue feature data IM1 and transmitted it to the control device 110. Next, in step S220, the control device 110 compares the current tissue feature data IM1 of the target skin 152 with the reference tissue feature data RIM in the reference database REFDB, and finds a reference tissue feature data matching with the current tissue feature data IM1 among the reference tissue feature data RIM, such as the reference tissue feature data RIM-1. The control device 110 takes a reference energy parameter corresponding to the reference tissue feature data RIM-1 as a suggested energy parameter REFP1 according to the first correspondence. The reference database REFDB may provide one or more suggested energy parameters to the control device 110, which is not limited in the present invention.

In addition, the control device 110 further compares the personal characteristics of the energy-release object 150 with the reference database REFDB, and obtains another reference energy parameter corresponding to the personal characteristics of the energy-release object 150 to serve as another suggested energy parameter REFPC.

In step S230, the control device 110 obtains the suggested energy parameter REFP1 and the suggested energy parameter REFPC from the storage device 130, and generates an energy parameter EP1 of an initial energy-release operation (a first energy-release operation) based on the suggested energy parameters.

In step S240, the energy-release apparatus 120 receives the energy parameter EP1 from the control device 110, and in step S242, the energy-release apparatus 120 performs the first energy-release operation on the target skin 152 according to the energy parameter EP1.

Since the different energy-release objects 150 may have different personal characteristics, such as different physiological conditions, different skin problems, etc., the feedback energy-release system 100 of the present embodiment may automatically generate different energy parameters for different energy-release objects 150 before the first irradiation operation. In this way, it will avoid the energy-release apparatus 120 from releasing inappropriate energy by a user only depending on his experience and increase user convenience through automated settings to enable efficient energy-release operations. However, in another embodiment, the control device 110 may also generate the energy parameter EP1 of the first energy-release operation based only on the energy suggestion parameter REFP1.

After the first energy-release operation is completed, proceeding to step S250 and step S252, the control device 110 receives the new tissue feature data IM2 of the target skin 152 by the feature capturing device 140. In step S254 and step S256, the control device 110 compares the tissue feature data IM2 with the reference tissue feature data RIM, for example, the reference tissue feature data RIM-2 matches the tissue feature data IM2. According to the first correspondence, the reference energy parameter corresponding to the reference tissue feature data RIM-2 can be used as another suggested energy parameter REFP2.

In step S260, the control device 110 may observe changes in tissue feature data before and after the first energy-release operation (i.e., compare the tissue feature data IM1 with the tissue feature data IM2), and generate the energy parameter EP2 of the next energy-release operation according to the difference between the tissue feature data IM1 and the tissue feature data IM2 and the suggested energy parameter REFP2. The suggested energy parameter REFP2 will be transmitted to the energy-release apparatus 120 for the next energy-release operation, as shown in steps S262 and S264.

After the second energy-release operation (e.g., step S264) is completed, proceed to step S270 and step S272, the feature capturing device 140 transmits the current tissue feature data IM3 of the target skin 152 to the control device 110. In the step S274 and step S276, the control device 110 compares the tissue feature data IM3 with the reference tissue feature data RIM, and the reference tissue feature data RIM-3 matching the tissue feature data IM3 as an example. According to the first correspondence, the reference energy parameter corresponding to the reference tissue feature data RIM-3 can be used as another suggested energy parameter REFP3.

In step S280, the control device 110 compares the tissue feature data captured before and after the second energy-release operation (step S264) to obtain a difference between the tissue feature data IM2 and the tissue feature data IM3. The energy parameter EP3 of the third energy-release operation is generated according to the difference and the suggested energy parameter REFP3. The energy parameter EP3 is transmitted to the energy-release apparatus 120 for a third energy-release operation (as shown in steps S282 and S284). By analogy, the above steps are repeated until the control device 110 causes the energy-release apparatus 120 to stop releasing energy E when the control device 110 determines that the target skin 152 has reached a suitable reaction state or is unsuitable for further energy-release operations (step S290).

In another embodiment, besides according to the difference in the tissue feature data before and after the last energy-release operation and the suggested energy parameters corresponding to the latest tissue feature data, the control device 110 may refer to the suggested energy parameter REFPC corresponding to personal characteristics to generate the energy parameter. In another embodiment, the sequence of steps for obtaining suggested energy parameters and comparing tissue feature data before and after the energy-release operation may be exchanged. In another embodiment, the control device 110 may also generate the next energy parameter only according to the difference between the two tissue feature data before and after the energy-release operation or the suggested energy parameters, which is not limited by the present invention.

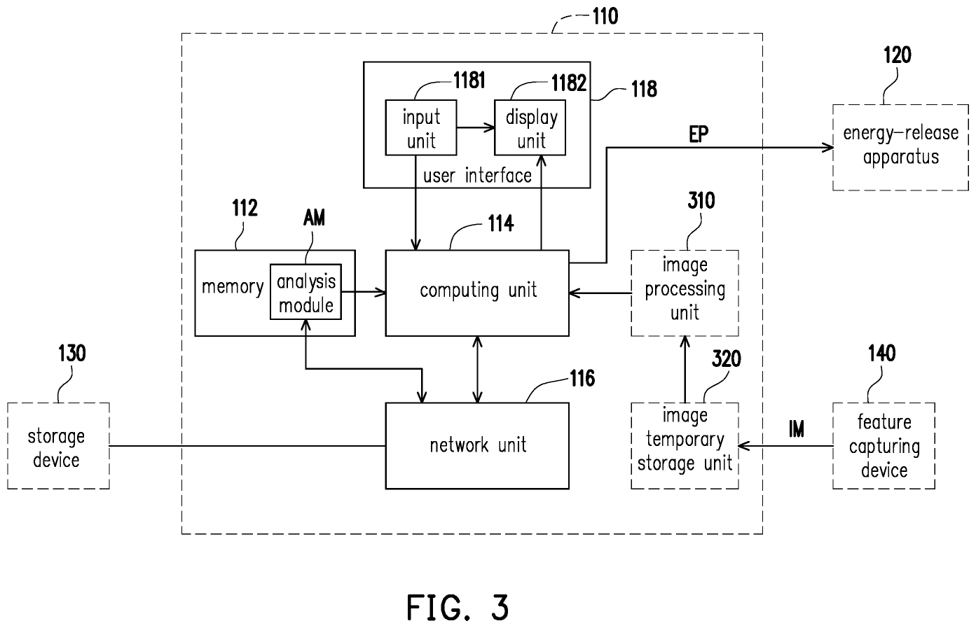

FIG. 3 is a block diagram illustrating a control device according to the embodiment of FIG. 1 of the present invention, which may be adapted to the feedback energy-release system 100 in FIG. 1 and FIG. 2. Referring to FIG. 3, the control device 110 includes a memory 112, a computing unit 114, a network unit 116, and a user interface 118. In the present embodiment, the control device 110 is connected to the storage device 130 through the network unit 116, and the memory 112 stores the analysis module AM. The computing unit 114 is coupled to the memory 112, the network unit 116 and the user interface 118. The computing unit 114 accesses the analysis module AM of the memory 112 to generate an energy parameter EP.

In the embodiment, the computing unit 114 is, for example, a central processing unit (CPU), or another programmable microprocessor, a digital signal processor (DSP), and a programmable controller, an application specific integrated circuit (ASIC), a programmable logic device (PLD), or other similar devices. The present invention is not limited thereto.

The memory 112 is, for example, any type of stationary or mobile random access memory (RAM), a read-only memory (ROM), a flash memory, a hard disk, or other similar devices, or an integrated circuit or combinations thereof. In addition to storing the analysis module AM, the memory 112 stores other data, codes, images that may be used in the operation of the feedback energy-release system 100. That is, the memory 112 is further used to record a plurality of instructions executed by the computing unit 114.

The network unit 116 is, for example, a wireless network card supporting wireless communication standards such as 802.11n/b/g of the Institute of Electrical and Electronics Engineers (IEEE) or a wire network card supporting wire communication standards such as Ethernet. The network unit 116 can be connected to other devices on the network by wire or wirelessly.

The user interface 118 includes, for example, an input unit 1181 and a display unit 1182. For example, a user can input information by using a keyboard, touch, image or voice input method through the user interface 118. The information is about the energy-release object 150, such as past energy release processing records or processing results, or personal characteristics. The user also may input the adjustment parameters through the user interface 118. The computing unit 114 receives the adjustment parameter from the input unit 1181 and adjusts the energy parameter EP according to the adjustment parameter. The display unit 1182 displays the medical record data or personal characteristics of the energy-release object 150, the received tissue feature data IM, or the reference tissue feature data RIM corresponding to the tissue feature data IM.

For example, the control device 110 receives the suggested energy parameter REFP2, and the control device 110 can base on the difference between the tissue feature data IM1 and the tissue feature data IM2 to determine whether the suggested energy parameter REFP2 is appropriate through the analysis module AM or further adjust the suggested energy parameter REFP2 to generate the energy parameter EP2. Alternatively, the control device 110 may calculate a set of energy parameters by the analysis module AM according to the difference between the tissue feature data IM1 and the tissue feature data IM2. In another embodiment, the memory 112 of the control device 110 can store a lookup table, and the control device 110 can obtain the reference energy parameters from the lookup table based on the difference between the tissue feature data IM1 and the tissue feature data IM2.

Next, the control device 110 may generate the energy parameter EP2 according to the reference energy parameters and the suggested energy parameter REFP2. Those skilled in the art can make appropriate adjustments of how to generating the energy parameter based on the above description and general knowledge.

In an embodiment, the tissue feature data IM includes image data, thus the feedback energy-release system 100 may further include an image processing unit 310 and an image temporary storage unit 320. The image processing unit 310 and the image temporary storage unit 320 may be disposed of in the feature capturing device 140 or the control device 110. In the embodiment, the image processing unit 310 and the image temporary storage unit 320 are disposed inside the control device 110. The image processing unit 310 can be executed in a hardware or software manner, such as an image processor or in an electrical circuit, or can be executed by the computing unit 114 executing image processing instructions. The image temporary storage unit 320 is, for example, a register or a memory.

The image processing unit 310 is coupled between the image temporary storage unit 320 and the computing unit 114, wherein the image temporary storage unit 320 is coupled to the feature capturing device 140 for temporarily storing the tissue feature data IM from the feature capturing device 140. After accessing the tissue feature data IM from the image temporary storage unit 320, the image processing unit 310 performs image process on the image data of the tissue feature data IM to determine the image feature. For example, the image processing unit 310 can convert the original chromaticity space of the image into another chromaticity space, so as to obtain the image feature therein, such as grayscale values in each pixel in the image or monochrome pixel values to obtain a graphic outline on the surface of the tissue. Thus, the computing unit 114 can determine the color block, color depth and area of the image, wherein a color block can determine the image characteristics. The image process is not limited by specific color spaces, such as RGB (red, green, blue), HSV (hue, saturation, value), CMYK (cyan, magenta, yellow, black), etc. The image processing unit 310 may also select to mark the specific position on the image, so that the user can easily recognize the image feature, for example, by adding a ghost shadow. The captured image may be a visible light image, an infrared thermal image, or an ultrasonic image.

In one embodiment, the image feature includes, for example, telangiectasia, hemangioma, varicose veins, etc. Alternatively, the image feature may include freckles, sunburn, age spots, black sputum, the signs of increased or decreased pigmentation, scars, wrinkles, lines, pores, hair, structural thickness variation of the skin epidermis or dermis layer, and/or externally added pigments (e.g., tattoos).

In an embodiment, the computing unit 114 receives the processed image from the image processing unit 310 to determine whether there is a matching reference tissue feature data RIM according to the image feature, or compare the difference of the target skin 152 before and after the energy-release operation according to the image feature.

In an embodiment, the computing unit 114 may determine the energy parameter EP by artificial intelligence (AI), wherein the artificial intelligence refers to a machine learning method, such as a multi-resolution convolutional neural network for training deep learning model. The analysis module AM is, for example, a machine learning module, which is trained by the tissue feature data IM, the difference between the tissue feature data before and after each of the previous energy-release operations, and the suggested energy parameters or the used energy parameter, so as to estimate the effect of the next energy-release operation and generate the energy parameter EP of the next energy-release operation.

Since the control device 110 is equipped with a machine learning module, after accumulating sufficient trainings with the tissue feature data IM and the corresponding energy parameters EP, the computing unit 114 performs the analysis module AM to generate the energy parameter EP of the next energy-release operation in a short period of time based on the previous tissue feature data IM.

In an embodiment, the feature capturing device 140 may capture the tissue feature data IM at different time points of each energy-release operation, such as capturing the tissue feature data IM before the energy-release operation, capturing the tissue feature data IM while the target skin 152 being irradiated by the energy E during the energy-release operation, and capturing the tissue feature data IM after the energy-release operation. The tissue feature data IM may include a plurality of different types of skin feature data, such as at least two types of image data, at least two types of skin physiological data, or a combination of at least one image data and at least one skin physiological data. The types of image data are, for example, optical images, infrared thermal images, ultraviolet light images, OCT images, or ultrasonic images, and the types of skin physiological data are, for example, moisture content or oil content of the skin, pH value or temperature, etc. The present invention does not limit the type of image data and the type of skin physiological data.

The control device 110 divides all types of skin feature data into several grades and determines the grade of each of the received skin feature data. The control device 110 records a grade distribution of the tissue feature data IM, the energy parameter EP used at the time and the results and extent of the skin improvement or deterioration condition. In an embodiment, the feature capturing device 140 photographs the skin images before and after the energy-release operation and also keeps tracking moisture content of the target skin 152. The control device 110 may determine the grade of each skin image based on the grayscale and records the grayscale variation of the target skin 152 over time. The control device 110 may also divide the moisture content into multiple ranges and determining the moisture content level of the target skin 152.

The control device 110 observes whether the moisture content changes its level during the energy-release operation and records the result. Herein, the grayscale variation of the skin images and the moisture content over time are referred as the grade distribution of the tissue feature data IM.

The control device 110 analyzes the correlation between the grade distribution of the tissue feature data IM and the energy parameter EP by multiple trainings. The control device 110 can determine the weighting value of each skin feature data according to a grade trend of each skin feature data in the plurality of energy-release operations, and thus the control device 110 can further determine which type of skin feature information is chosen as the main basis (maybe with the higher weighting value) for determining the energy parameter EP of the next energy-release operation according to the weighting values of the skin feature data.

It should be noted that, in another embodiment, the tissue feature data IM may include only optical images, infrared thermal images, ultraviolet images, OCT images, skin physiological detection data, or ultrasound images. The present invention does not limit the number of types of skin profile data included in the tissue feature data IM.

In an embodiment, the control device 110 can also feed the received tissue feature data

IM, the calculated energy parameter EP, or the personal characteristics of the energy-release object 150 to the storage device 130 through the network unit 116 to update the reference database REFDB.

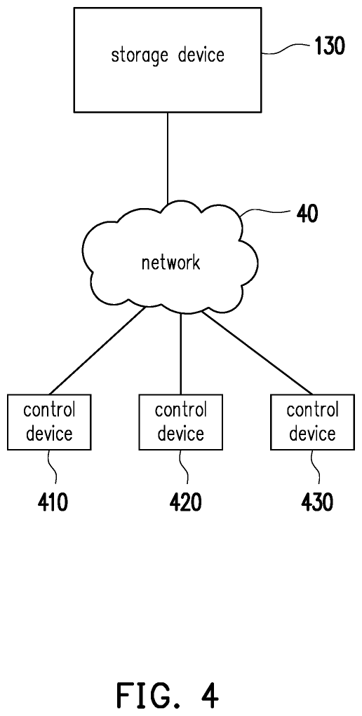

FIG. 4 is a schematic diagram illustrating a network architecture of a feedback energy-release system in accordance with an embodiment of the present invention. In the embodiment, the storage device 130 is a cloud storage device, so multiple control devices can be connected to the storage device 130 through the network 40. Only three control devices 410, 420, and 430 are shown in FIG. 4, but the present invention is not limited to three. The storage device 130 can collect statistical data about personal characteristics, tissue feature data, and energy parameters through the network 40 to update the first correspondence and the second correspondence. In addition, the storage device 130 can also synchronize all published medical journals or other information sources on the network to create newer and more complete reference energy parameters corresponding to the tissue feature data. Therefore, the control device 110 having the above-mentioned machine learning capability can use the reference database REFDB or the processed reaction result as machine learning data with the big data analysis when connecting to the storage device 130.

In other embodiments, the control device 110 can also be a cloud server and perform the big data analysis based on data collected by the storage device 130. Since training the deep learning model may require a higher performance processor for analysis, the cloud server can perform calculations and then provide energy parameters to the energy-release apparatus 120 to reduce the burden of the user's hardware computing requirements.

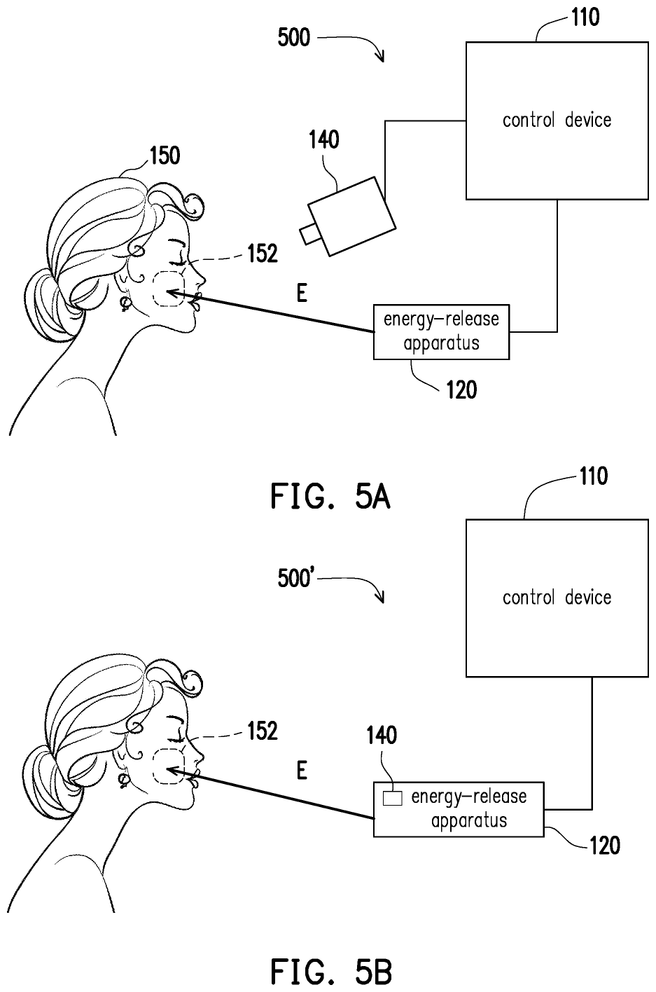

FIG. 5A is a schematic diagram illustrating a feedback energy-release system in accordance with another embodiment of the present invention. FIG. 5B is a schematic diagram illustrating a feedback energy-release system in accordance with another embodiment of the present invention. The feedback energy-release system 500 of FIG. 5A and the feedback energy-release system 500′ of FIG. 5B both include the energy-release apparatus 120 in addition to the feature capturing device 140 and the control device 110 and are also applicable to the feedback energy-release system 100 of FIG. 1 through FIG. 4. In the embodiment of FIG. 5A, the energy-release apparatus 120 and the feature capturing device 140 may be two separate devices that are coupled to the control device 110 by wire or wirelessly, respectively. In the embodiment of FIG. 5B, the feature capturing device 140 can be integrated into the energy-release apparatus 120. The feature capturing device 140 can simultaneously capture tissue feature data IM during each energy-release operation to monitor the effect and adjust the energy released by the energy-release apparatus 120 in real time. When the control device 110 determines that the target skin 152 is not suitable for receiving the energy E anymore, the energy-release apparatus 120 stop releasing the energy E.

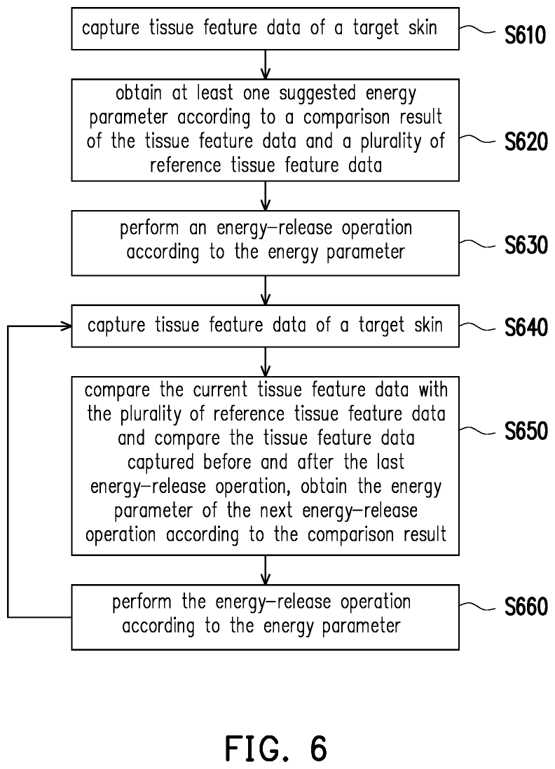

FIG. 6 is a flow chart illustrating an operation method of the feedback energy-release system in accordance with an embodiment of the present invention. The operation method of the feedback energy-release system of FIG. 6 is applicable to the embodiment of the feedback energy-release system of FIG. 1 through FIG. 5B described above. In step S610, the tissue feature data of a target skin are captured, and in step S620, a plurality of reference energy parameters have the first correspondence with a plurality of reference tissue feature data, so the feedback energy-release system can compare the tissue feature data with the plurality of reference tissue feature data to obtain at least one suggested energy parameter. In an embodiment, besides comparing the tissue feature data with the plurality of reference tissue feature data, the feedback energy release system can further obtain a reference energy parameter according to a second correspondence to set as another suggested energy parameter corresponding to personal characteristics of the energy-release object 150, wherein the plurality of reference energy parameters have the second correspondence with personal characteristics. The feedback energy-release system can generate an energy parameter based on the suggested energy parameters described above.

Next, in step S630, the energy-release apparatus performs an energy-release operation on the target skin according to the energy parameter. After the energy-release operation is completed, then proceed to step S640 to capture the tissue characteristics of the target skin again. Next, in step S650, according to the difference between the tissue feature data captured before and after the last energy-release operation (the energy-release operation in step 630) and the comparison result of the current tissue feature data (the tissue feature data in step 640) and the above-mentioned reference tissue feature data, the energy parameters of the next energy-release operation is generated. In step S660, the energy-release apparatus performs another energy-release operation on the target skin according to the energy parameter obtained in step S650. Step S640 is then re-executed to obtain the new energy parameter of the next energy-release operation.

The related component features and specific embodiments of the operation method of the feedback energy-release system of the present embodiment can be sufficiently taught, suggested, and implemented from the description of the embodiment of FIG. 1 to FIG. 5B, and therefore will not be described again.

In summary, the feedback energy-release system and the operation method thereof in the embodiments of the present invention include a feature capturing device and a control device.

The feature capturing device captures a plurality of tissue feature data of an energy-release object. The control device obtains a suggested energy parameter in accordance with a first correspondence based on the comparison result of the tissue feature data and a plurality of reference tissue feature data, wherein the first correspondence is a relationship between the plurality of reference tissue feature data and a plurality of reference energy parameters. The control device compares the tissue feature data before and after the current energy-release operation and generates the energy parameter of the next energy-release operation according to the comparison result and the suggested energy parameter. The feedback energy-release system can compare the current tissue feature data with the plurality of reference tissue feature data to obtain an objective suggested energy suggestion parameter and further adjust the energy parameter of the next energy-release operation according to the result of the previous energy-release operation. Thereby, the feedback energy-release system enables the user to reduce the risk of operational errors and effectively improve the effect of the energy-release operation.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A feedback energy-release system for provide an energy parameter to an energy-release apparatus, wherein the energy-release apparatus performs a plurality of energy-release operations on a target skin of an energy-release object according to the energy parameter, the feedback energy-release system comprising:
a feature capturing device, configured to capture a plurality of tissue feature data of the target skin at different time points during each of the plurality of energy-release operations, wherein the tissue feature data includes a first tissue feature data and a second tissue feature data which are respectively captured before and after a current energy-release operation, and the tissue feature data includes a plurality of different types of skin feature data; and
a control device, coupled to the feature capturing device and the energy-release apparatus, and configured to:
receive the tissue feature data from the feature capturing device;
determine grades of the tissue feature data of the current energy-release operation for each type of the skin feature data;
record a grade distribution of the tissue feature data generated using the grades of the tissue feature data of the current energy-release operation, energy parameter of the current energy-release operation, and skin condition after the current energy-release operation;
determine a weighting value of each of the skin feature data according to a grade trend determined from the grade distribution of each of the skin feature data; and
determine and generate the energy parameter of a next energy-release operation subsequent to the current energy-release operation according to a difference between the first tissue feature data and the second tissue feature data, the grade distribution, and the weighting values of the plurality of skin feature data; and
transmit a control signal including the generated energy parameter to the energy-release apparatus for performing the next energy-release operation on the target skin according to the generated energy parameter.

2. The feedback energy-release system according to claim 1, further comprising:
a reference database, coupled to the control device, storing a plurality of reference energy parameters and a plurality of reference tissue feature data corresponding to the reference energy parameters, wherein the plurality of reference energy parameters have a first correspondence with the plurality of reference tissue feature data,
wherein the control device is further configured to
compare the second tissue feature data with the plurality of reference tissue feature data to obtain a reference tissue feature data matching the second feature data among the plurality of reference tissue feature data;
provide the reference energy parameter corresponding to the matched reference tissue feature data as at least one suggested energy parameter,
wherein the energy parameter of the next energy-release operation is determined and generated further according the at least one suggested energy parameter.

3. The feedback energy-release system according to claim 2, wherein, before the target skin receives any of the energy-release operations, the control device compares the current tissue feature data of the target skin with the plurality of reference tissue feature data to obtain the at least one suggested energy parameter according to the first correspondence, and the control device generates the energy parameter of an initial energy-release operation according to the at least one suggested energy parameter.

4. The feedback energy-release system according to claim 3, wherein the plurality of reference energy parameters have a second correspondence with personal characteristics, wherein the personal characteristics comprise at least one of race, gender, age, skin color, skin type, place of residence, degree of sun exposure, medication record, type of skin problems, extent of skin problems, treatment of skin problems and reaction of the treatment; and
the at least one suggested energy parameter includes a plurality of suggested energy parameters,
wherein according to the second correspondence, the control device further obtains, from the reference database, the reference energy parameter corresponding to the personal characteristics of the energy-release object as one of the plurality of suggested energy parameters.

5. The feedback energy-release system according to claim 4, wherein the control device feeds the received tissue feature data, the energy parameter or the personal characteristics of the energy-release object to the reference database to update at least one of the reference energy parameters, the reference tissue feature data, the first correspondence, and the second correspondence.

6. The feedback energy-release system according to claim 1, wherein the energy parameter comprises at least one of intensity, a wavelength, a waveform, a bandwidth, a length of energy release time, density, an energy spot size, a region where the target skin receives the energy, and a depth of the energy penetrating the target skin, and the energy comprises at least one of a laser, an electromagnetic wave, an ultrasonic wave, and a pulsed light; and
wherein the control device makes the energy-release apparatus stop releasing the energy when the control device determines that the target skin is unsuitable for receiving the energy.

7. The feedback energy-release system according to claim 1, wherein the control device comprises a machine learning module, wherein the machine learning module is trained by using the tissue feature data of a plurality of previous energy-release operations and the corresponding energy parameter used in each of the plurality of previous energy-release operations,
wherein the control device is further configured to generate the energy parameter of the next energy-release operation by inputting the tissue feature data of the current energy-release operation to the machine learning module.

8. The feedback energy-release system according to claim 1, wherein the control device is further configured to retrain a machine learning module by using the tissue feature data of the current energy-release operation to the machine learning module,
wherein the machine learning module is pre-trained by using the tissue feature data of a plurality of previous energy-release operations and the corresponding energy parameter used in each of the plurality of previous energy-release operations.

9. The feedback energy-release system according to claim 1, wherein the plurality of different types of skin feature data comprises at least two types of image data, at least two types of skin physiological data or a combination of at least one image data and at least one skin physiological data.

10. An operation method of a feedback energy-release system, adapted to provide an energy parameter to an energy-release apparatus to perform each of a plurality of energy-release operations on a target skin of an energy-release object, the operation method comprising:
capturing a plurality of tissue feature data of the target skin at different time points during each of the plurality of energy-release operations, wherein the tissue feature data includes a first tissue feature data and a second tissue feature data respectively captured before and after each of the energy-release operations, and the tissue feature data includes a plurality of different types of skin feature data;
determining grades of the tissue feature data of a current energy-release operation for each type of the skin feature data and recording a grade distribution of the tissue feature data generated using the grades of the tissue feature data of the current energy-release operation;
determining a weighting value of each of the skin feature data according to a grade trend of determined from the grade distribution of each of the skin feature data;
determining and generating the energy parameter of a next energy-release operation according to a difference between the first tissue feature data and the second tissue feature data, the grade distribution, and the weighting values of the plurality of skin feature data; and
transmitting a control signal including the generated energy parameter to the energy-release apparatus for performing the next energy-release operation on the target skin according to the generated energy parameter.

11. The operation method according to claim 10, wherein the step of comparing the second tissue feature data with the plurality of reference tissue feature data to obtain the at least one suggested energy parameter comprising:
finding a reference tissue feature data matching with the second tissue feature data among the plurality of reference tissue feature data and providing a reference energy parameter corresponding to the matched reference tissue feature data as at least one suggested energy parameter, wherein a plurality of the reference energy parameters have a first correspondence with the plurality of reference tissue feature data; and
further generating the energy parameter of the next energy-release operation based on the at least one suggested energy parameter.

12. The operation method according to claim 11, further comprising:
before the target skin receives any of the energy-release operations, comparing the current tissue feature data of the target skin with the plurality of reference tissue feature data to obtain the at least one suggested energy parameter according to the first correspondence; and
generating the energy parameter of an initial energy-release operation according to the at least one suggested energy parameter.

13. The operation method according to claim 12, wherein the step of generating the energy parameter of the initial energy-release operation according to the at least one suggested energy parameter comprising:
the plurality of reference energy parameters having a second correspondence with personal characteristics, wherein the personal characteristics comprise at least one of race, gender, age, skin color, skin type, place of residence, degree of sun exposure, medication record, type of skin problems, extent of skin problems, treatment of skin problems and reaction of the treatment;
the at least one suggested energy parameter including a plurality of suggested energy parameters; and
according to the second correspondence, further obtaining the reference energy parameter corresponding to the personal characteristics of the energy-release object as one of the plurality of suggested energy parameters.

14. The operation method according to claim 13, further comprising:
feeding the received tissue feature data, the energy parameter or the personal characteristics of the energy-release object to a reference database to update at least one of the reference energy parameters, the reference tissue feature data, the first correspondence, and the second correspondence.

15. The operation method according to claim 10, wherein the energy parameter comprises at least one of intensity, a wavelength, a waveform, a bandwidth, a length of energy release time, density, an energy spot size of the energy released by the energy-release apparatus, a region where the target skin receives the energy, and a depth of the energy penetrating the target skin, and the energy comprises at least one of a laser, an electromagnetic wave, an ultrasonic wave, and a pulsed light; and
the operation method further comprising:
making the energy-release apparatus stop releasing the energy when the target skin is determined to be unsuitable for receiving the energy.

16. The operation method according to claim 10, wherein the step of generating the energy parameter of the next energy-release operation further comprising:
inputting the tissue feature data of the current energy-release operation into a machine learning module to generate the energy parameter of the next energy-release operation, wherein the machine learning module is pre-trained by using the tissue feature data of a plurality of previous energy-release operations and the corresponding energy parameter used in each of the plurality of previous energy-release operations.

17. The operation method according to claim 10, further comprising'
retraining a machine learning module by using the tissue feature data of the current energy -release operation to the machine learning module, wherein the machine learning module is pre-trained by using the tissue feature data of a plurality of previous energy-release operations and the corresponding energy parameter used in each of the plurality of previous energy-release operations.

18. The operation method according to claim 10, wherein the plurality of different types of skin feature data comprises at least two types of image data, at least two types of skin physiological data or a combination of at least one image data and at least one skin physiological data.
